(19) United States
(12) Patent Application Publication
(10) Pub. No.: US 2012/0101157 A1
(43) Pub. Date: Apr. 26, 2012

(54) 4,5-Diamino-3-Halo-2-Hydroxybenzoic Acid Derivatives and Preparations Thereof

(75) Inventors: AN-RONG LEE, TAIPEI CITY (TW); WEN-HSIN HUANG, TAIPEI CITY (TW); CHI-HONG CHU, TAIPEI CITY (TW); WEN-LIANG CHANG, TAIPEI CITY (TW); CHEN-WEN YAO, TAIPEI CITY (TW); I-LING CHEN, TAIPEI CITY (TW)

(73) Assignee: NATIONAL DEFENSE MEDICAL CENTER, Taipei City (TW)

(21) Appl. No.: 13/279,519

(22) Filed: Oct. 24, 2011

Related U.S. Application Data
(60) Provisional application No. 61/438,082, filed on Jan. 31, 2011.

(30) Foreign Application Priority Data
Oct. 25, 2010 (TW) 099136373

(51) Int. Cl. A61K 31/245; C07C 233/54; C07C 231/14; A61P 31/16; C07C 277/08; C07C 279/18; A61K 31/196

(52) U.S. Cl. 514/535; 560/34; 560/46; 562/439; 562/453; 514/563

(57) ABSTRACT

Disclosed are 4,5-diamino-3-halo-2-hydroxybenzoic acid derivatives and manufactures thereof. The 4,5-diamino-3-halo-2-hydroxybenzoic acid derivatives are presented by formula (I):

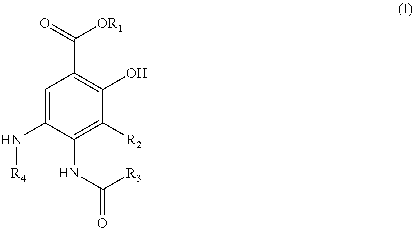

wherein R₁ group is H, CH₃, or C₂H₅; R₂ group is H, or Br; R₃ group is CH₃, or C₃H₇; and R₄ group is H, or C(═NH)—NH₂. 4,5-diamino-3-halo-2-hydroxybenzoic acid derivatives provided here were non-toxic to MDCK cells, particularly compounds 6a, 6b, 6c, 6e, 6f, 7a, 7b and 8 had better anti-H1N1 activity. In the future, these compounds can be used to focus on viral neuraminidases as targets to develop effective anti-influenza drugs.

### BACKGROUND OF THE INVENTION

1. Field of the Invention

The present invention relates to compounds against influenza viruses. More particularly, the present invention relates to 4,5-diamino-3-halo-2-hydroxybenzoic acid derivatives as influenza neuraminidase inhibitors.

2. The Prior Arts

Influenza, also referred to as “flu”, is an acute respiratory disease caused by influenza viruses. Due to its high antigenic variability and rapid-spreading, influenza has caused several global pandemics that severely harmed the economy and human health. In the 20^{th} century, there were three influenza pandemics occurred in 1918 (“Spanish” influenza, H1N1), in 1957 (“Asian” influenza, H2N2), and in 1968 (“Hong kong” influenza, H3N2). In particular, the pandemic occurred during 1918-1919 was the most severe one and caused more than 40,000,000 deaths. The first time H5N1 avain influenza infections occurred in Hong Kong (1997), was of great concerns in the world, because of its high death rate, human-to-human transmission, and drug resistance to commercial anti-influenza drugs. Therefore, it was an important object to develop an effective drug to prevent the next global epidemic. As known by now, neuraminidases are the one of the important surface glycoproteins of the influenza virus, and are significantly associated with their replication and infectious ability. Because of the active sites of neuraminidases are highly conserved in influenza types A and B, neuraminidases are considered as a potential target in anti-influenza drug design.

### I. Influenza Types

Influenza viruses are negative-sense single-stranded RNA viruses, and belong to the family Orthomyxoviridae, can be classified to type A, B and C according to their nucleoprotein and matrix protein antigenicity.

### (i) Influenza A Virus

Influenza A viruses belong to Influenzavirus A genus and have a great variety of host species including the human, pig, horse, marten, whale, chicken, duck, and goose etc. Due to highly antigenic variability, Influenza A viruses have caused several large scale pandemics in the past. Influenza A viruses can be classified to different subtypes according to two surface antigens that include hemagglutinin (HA) and neuraminidase (NA). The hemagglutinin has 16 different subtypes (H1-H16), and the neuraminidase has 9 different subtypes (N1-N9). All subtypes can be found in birds, however, there are only three HA subtypes (H1, H2 and H3) and two NA subtypes (N1 and N2) can be transmitted among people. Because all subtypes of influenza A viruses can be transmitted between wild fowls, which are thus referred to as the natural hosts of influenza A viruses. Influenza transmitted among birds is so called “avian influenza”. Normally, avian influenza viruses do not directly transmit from birds to humans or humans to humans, but only among birds. Nevertheless, H₅N₁, H₇N₇ and H₉N₂ have been found that they can be transmitted from other species to humans so far.

### (ii) Influenza B Virus

Influenza B viruses belong to Influenzavirus B genus and have only one host species, the human. The influenza B virus has only one type of hemagglutinin and neuraminidase, so that it has a lower antigenic variety and only can cause regional epidemics, instead of large scale pandemics.

(iii) Influenza C Virus

Influenza C viruses belong to Influenzavirus C genus and have two host species including the human and pig. Influenza C viruses hardly result in influenza and epidemics, symptoms caused by them are mostly mild.

### II. Structure of Influenza Virus

With reference to FIG. 1, it is shown that the structure of an influenza virus. Influenza viruses have multiple conformations and are enveloped by lipid envelopes generated from hosts. For the spherical virus, it has a diameter of 100 nm, and the rod-shaped virus has a diameter of more than 300 nm. The lipid membrane of an envelope is coated with spike-shaped glycoproteins. As far as influenza A and B viruses, they have two major glycoproteins: hemagglutinin (HA) and neuraminidase (NA). Influenza C viruses merely have one type of surface glycoprotein: hemagglutinin-esterase-fusion (HEF) protein combined with both functions of the hemagglutinin and neuraminidase. Additionally, there are several secondary proteins acting as ion channels which include the M2 protein (influenza A virus), BM2 protein (influenza B virus), and CM2 protein (influenza C virus). Furthermore, influenza B viruses have a unique protein: the NB protein, which is structurally similar to ion channel proteins and not a non-essential protein in viral replication, its actual function is not clear so far. Beneath the lipid membrane is a viral protein called matrix protein (M1), which forms a shell and gives strength and rigidity to the lipid envelope, so that the ribonucleoprotein (RNP) can be well protected, and the M1 protein also plays a crucial role while being released from the host cell after replication. Within the interior of the virion are the viral RNAs, 8 of them for influenza A viruses and 7 of them for influenza B viruses. These are the genetic material of the virus, they code for one or two proteins. Each RNA segment consists of RNA joined with several proteins: B1, PB2 and PA proteins (forming RNA polymerase) and nucleoprotein (NP). These RNA segments are the genes of influenza virus. The interior of the virion also contains another protein called the nonstructural protein: (nuclear export protein, NEP/NS2) which plays a important role while viral RNP transporting from host cell nucleus to cytoplasm.

### III. Surface Glycoprotein of Influenza Virus

There are two glycoproteins on the surface of influenza virus particles: the hemagglutinin and neuraminidase, they are significantly associated with the viral antigenicity. These two antigen proteins are described as follow.

### (i) Hemagglutinin (HA)

Influenza viruses recognize receptors on the surface of host cells by their hemagglutinins In addition, hemagglutinins are associated with the fusion of virus lipid envelopes and host cell membranes. As to the structure, the influenza virus hemagglutinin (HA) protein is translated in cells as a single protein (HA0), or hemagglutinin precursor protein, with 550 amino acids. The hemagglutinin precursor protein (HA0) can be cleaved by a trypsin-like serine endoprotease at a specific site. After cleavage, two disulfide-bonded protein domains produce the mature form of the protein subunits HA1 and HA2 which are linked by a disulfide-bond forming between cys-14 (HA1) and cys-137 (HA2). Structurally, a hemagglutinin can be divided into a spherical head and a stem-like structure consisting of 3α-helixes. The spherical head consists of HA1 only and has the receptor-binding domain used to bind to sialic acid (receptor) of host cells. A stem-like structure consists of HA2, and its C terminal is anchored in the host cell membrane and the N terminal is a part of fusion peptide. When viruses enter host cells by endocytosis, due to that the pH within the endosome drops to about 6.0, the original folded structure of the HA0 molecule is cleaved into two subunits HA1 and HA2. Therefore, the buried fusion peptide of HA2 is exposed and inserts into the host cell membrane to cause the fusion of the virus envelope and the host cell membrane (refer to FIG. 2). The hemagglutinin is also regarded as an N-glycosylated protein having a lot of glycosylatable sites, also referred to as antigen sites, whose antigenicity is associated with ability of the virus to recognize host cells.

### (ii) Neuraminidase (NA)

Neuraminidases are on the surface of influenza viruses that enable the viruses to be released from host cells. After viral replication, neuraminidases can cleave sialic acid groups from glycoproteins to enable viruses exit host cells to further infect other host cells. As similar as hemagglutinins, neuraminidases are an N-glycosylated protein, whose surface antigen sites are associated with its antigenicity. With reference to FIG. 3, neuraminidases are a tetramer composed of four identical polypeptides (monomer), include a highly conserved cytoplasmic tail and a hydrophobic transmembrane region which involves a stalk domain and a head domain, and the N terminal of the stalk domain serves to anchor in its envelope. Each monomer consists of six identical four-stranded antiparallel β-sheets and has both antigenic and enzymatic activity. Thus sialic acid can bind to each monomer's active site for catalysis process, and conformation of sialic acid is not changed during the process.

Influenza B viruses merely have one type of neuraminidase. For influenza A viruses, there are 16 neuraminidase subtypes and can be classified into two groups: group-1 and group-2. Group-1 includes N1, N4, N5 and N8 subtypes, and Group-1 includes N2, N3, N6, N7 and N9 subtypes. In comparison with group-2, group-1 has a “150-cavity” adjacent to its active site and extends its active site. There are two reasons that cause 150-cavity: (i) conformational difference between group-1 and group-2 centered on the 150-cavity: pointed away from the active site in group-1 but towards it in group-2; (ii) Glu 119 adopts a different conformation between the two groups. In group-2 structures Glu 119 residue forms a hydrogen bond with Arg 156, but not in group-1. This 150-cavity can provide a new direction for specificity in drug design. Basically, neuraminidase active sites between group-1 and group-2 are highly conserved (75% conserved) except that the active site in group-1 further has a 150-cavity. Compared with the hemagglutinin, the neuraminidase is an ideal target for drug design. There are two commercial drugs designed based on this mechanism, they are Oseltamivir and Zanamivir, respectively. As shown in FIG. 3, Oseltamivir can bind to the neuraminidase active site (as indicated by arrow).

### IV. Anti-Influenza Virus Agent

Because of the absence of RNA proofreading enzymes, virus structures and their antigenicity constantly change. Despite vaccines are the best way to combat against influenza, but the immunity induced by a vaccine is limited to a specific virus strain. Therefore, there is always short of an effective vaccine while breaking out an influenza pandemic caused by critical viral mutation. In this case, anti influence drugs are dependable.

### (i) M2 Inhibitor

The below two formulas as presented are adamantine derivatives which include amantadine and rimantadine. Rimantadine has fewer side effects, and amantadine is associated with several central nervous system side effects. Amantadine has been firstly approved as an anti-influenza drug merely effective against influenza virus A. The mechanism of Amantadine's antiviral activity involves interference with a viral protein (M2 ion channel), which can further inhibit the release of viral RNP. However, these drugs tend to generate resistant viruses and cause pandemics. In resent years, H3N2 has been found to show resistance to the adamantane and amantadine.

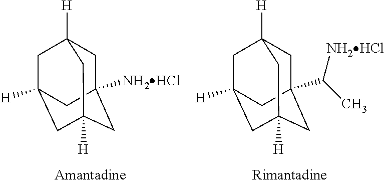

### (ii) Imp Dehydrogenase Inhibitor

Inosine 5′-monophosphate (IMP) dehydrogenase inhibitors include Ribavirin and Viramidine. Ribavirin is a widely used antiviral drug, also found to combat against influenza viruses. It is a prodrug of ribavirin in trials and expected to have better activity against influenza than Ribavirin. Ribavirin is a synthesized nucleotide analog and used to inhibit IMP dehydrogenase. IMP is catalyzed by IMP dehydrogenase, and finally forms GTP after a series of chemical reactions. Because GTP is associated with RNA synthesis, IMP dehydrogenase is commonly used to inhibit RNA virus synthesis. According to previous studies, EC₅₀ (50% effective concentration) of Ribavirin H5N1 is ranged from 6-22 μM, which shows inhibiting effects for H5N1.

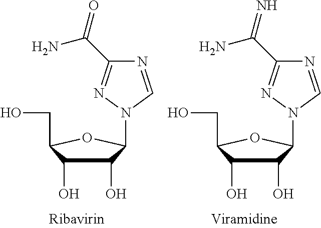

(iii) Neuraminidase Inhibitor

Two neuraminidase inhibitors, Oseltamivir and Zanamivir are shown as below formulas. Neuraminidase inhibitors cut the linkage between sialic acid residues and glycoproteins on host cell surface to inhibit release of virus particles. It is reported that neuraminidase inhibitors have a good inhibition ability for transition-state sialic acid during catalysis, and Oseltamivir and Zanamivir are the developed drugs based on this structure and they have good inhibition ability against influenza A and B (Oseltamivir: Kᵢ≈0.2 nM, Zanamivir: Kᵢ≈0.1 nM). Zanamivir is the first neuraminidase inhibitor commercially developed, and its dosing is limited to the inhaled route. Oseltamivir is an orally active neuraminidase inhibitor, also an ethyl ester prodrug, which is converted into its active form by esterase after it is taken into the body. So far, Oseltamivir is the most effective and widely-used anti-influenza drug.

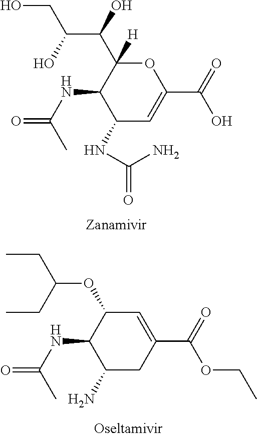

Unlike M2 inhibitors which work only against influenza A, have side effects and drug resistance, neuraminidase inhibitors act against both influenza A and influenza B and not tend to produce drug-resistant variants. Although Oseltamivir and Zanamivir are effective, newly developed drug design is also required, for example, neuraminidases are taken for a target. Neuraminidases play a crucial role as influenza viruses being released. It is further reported that glycoproteins of host cells are associated with virus spread in respiratory tracts. As known so far, active sites of neuraminidases between influenza A and B are highly conserved. Based on this feature, an effective anti-influenza drug may be developed and points a correct direction in drug design.

### Mechanism of Action of Neuraminidase

As shown in the below chemical reactions, which demonstrate the mechanism of neuraminidase function. As sialic acid binds to the active site of neuraminidase, electrostatic attraction formed between the carboxyl group of sialic acid and the enzymatic environment of neuraminidase, which results in the chair conformation of sialic acid is distorted to form the boat conformation. Due to this conformational strain, sialic acid is removed from the linked glycoprotein. In this catalysis process, a transition-state sialic acid (sialosyl cation) is firstly formed, and this structure can be stabilized within the negatively-charged active site. Then the water molecule reacts and the α-sialic acid is released, and it mutarotates to a stable β-sialic acid. According to studies, α-sialic acid is more active to react with neuraminidases in comparison with β-sialic acid. On the other hand, sialosyl cation can be captured by other neuraminidase to produce the glycosyl-enzyme intermediate (as shown below). It is reported that α-sialic acid, as an inhibitor, has weak inhibition ability. However, DANA (2-deoxy-2,3-didehydro-N-acetylneuraminic acid, Neu5Ac2en) has better inhibition efficacy (Kᵢ=4×10⁻⁶ M). Because of the transition state of sialic acid having better interaction with active sites of neuraminidases, a potential drug based on this structure with modification can be developed. Oseltamivir and Zanamivir are designed and developed according to this mechanism.

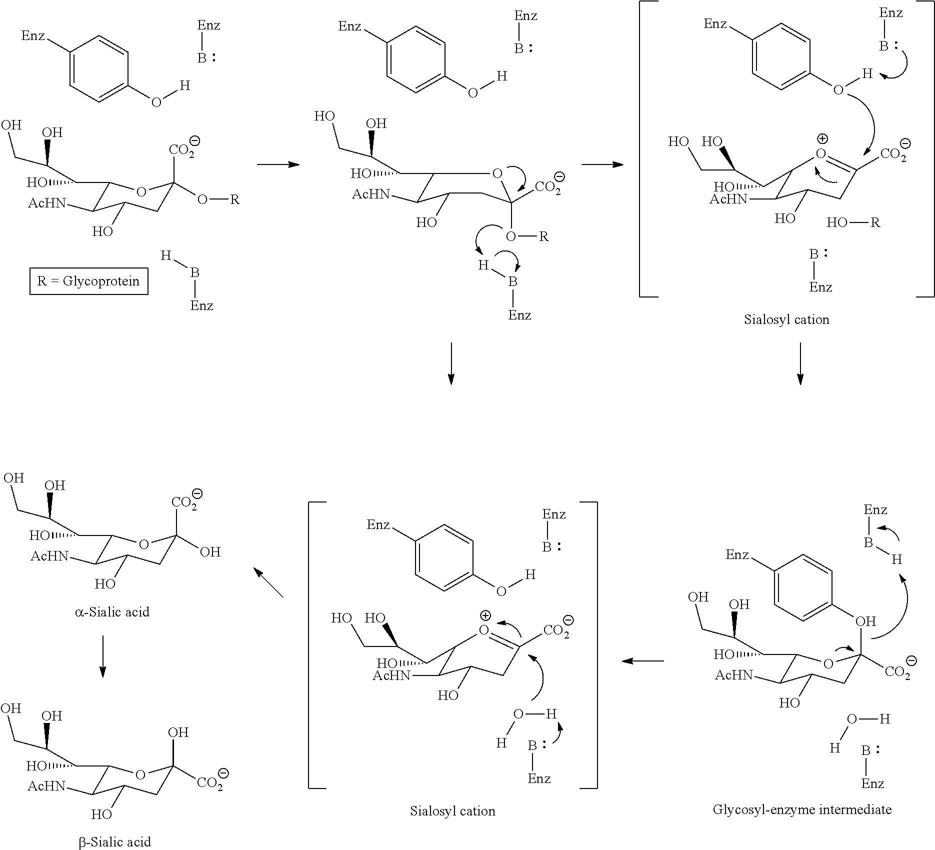

### Active Site of Neuraminidase

By computational protein structure analysis, interaction between active sites of neuraminidases and their substrates can be realized. Based on the article published by Stoll (Biochemistry, 2003), the active site is divided into 5 regions, termed subsites S1, S2, S3, S4 and S5. DANA (dehydro-deoxy-N-acetylneuraminic acid) is given to an example, interaction between the amino acids of the active site and the substrate is clearly described, and these five subsites are discussed as follow (refer to FIG. 4).

S1: Subsite S1 includes three residues, Arg 118, Arg 292, and Arg 371, they provide positively charged charge-charge interactions and hydrogen-bonding environment for anionic substituents from the inhibitor, such as carboxylate. This triarginyl cluster composed of three arginine residues also plays a crucial role in determining inhibitor orientation within the active site. In addition to carboxyl group, this residue can be substituted for other groups, such as phosphonic group (—PO(OH)₂), sulfonic group (—SO₂(OH)), or sulfinic group (—SO(OH)).

S2: Subsite S2 contains two glutamine residues, Glu 119 and Glu 227, they provide a negatively charged region of the active site. The hydroxyl group (C-4) of DANA has hydrogen bond interactions with these negatively charged residues. Due to its negatively charged environment, there is a stronger ionic interaction generated once the C-4 residue is substituted for positively charged moieties, such as amino group (—NH₂) and guanidino group (—NHC(NH₂)NH). Besides, an additional important active site residue adjacent to the subsite S2 is Asp151, and it is not formally part of the subsites as defined. According to the related studies, Asp 151 is believed to play a critical role in catalysis by polarizing the scissile glycosidic linkage.

S3: Subsite S3 contains a small hydrophobic region formed from the side chains of Trp 178 and Ile 222 and a hydrophilic region provided by the side chain of Arg 152 and a bound water molecule. The acetamide of DANA accepts an H-bond from Arg 152 and donates an H-bond to a water molecule. Traditionally, for several different inhibitors, they are used to add an acetamido group at this site, because this site substitutes for other hydrophobic moiety may strengthen interactions between inhibitors and enzymes.

S4 and S5: Subsite S4 is primarily a hydrophobic region derived from the side chains of Ile 222, Ala 246, and the hydrophobic face of Arg 224, and is not occupied by any portion of the DANA inhibitor. Subsite S5 is a region of mixed polarity and is comprised of the carboxylate of Glu 276 and the methyl of Ala 246. Glu 276 has a hydrogen bond interaction with the glycerol side chain of DANA, but also can exist in an alternative gauche conformation with its carboxylate ion-paired with Arg 224. When Glu 276 is in this conformation, its methylenes join with Ala 246 to create a hydrophobic pocket within S5. Therefore, adding a hydrophobic group at the site can increase its inhibiting ability.

### SUMMARY OF THE INVENTION

As described and discussed above, it is understood that a compound to mimic the transition state of sialic acid could be a potential inhibitor. In previous studies, compounds with modification in DANA side chains, for example, the structure with C4 hydroxyl group substituted with guanidine group has significant inhibition effects. However, this structure tends to be metabolized in the human body, so that it is not suitable to be developed. In this application, several derivatives based on DANA as major basic structures have inhibition effects. As shown below, it is exhibited that comparison of inhibiting ability of DANA and GBA (4-(acetylamino)-3-guanidino-benzoic acid) for influenza virus type A subtype H1N9 neuraminidases.

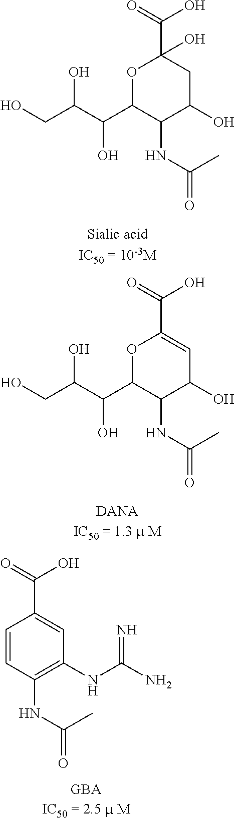

p-Aminosalicylic acid (PAS) is a second-line drug against tuberculosis, also used to be the initial reagent of the present invention. The outline of drug design of the present invention is exhibited as below: (i) the carboxyl group on C-1 is kept or converted to an ester group; (ii) the hydrogen atom on C-3 is kept or converted to lipophilic side chain or a bromo group; (iii) the amino group on C-4 site is converted to a amido group; and (iv) the hydrogen atom on C-5 site is converted to amino or guanidino group.

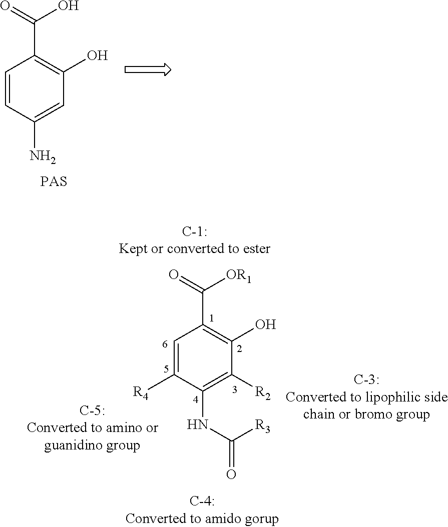

Therefore, the objective of the present invention is to provide 4,5-diamino-3-halo-2-hydroxybenzoic acid derivatives as influenza neuraminidase inhibitors and the manufacture thereof.

In one aspect, the present invention relates to a 4,5-diamino-3-halo-2-hydroxybenzoic acid derivative, as presented by formula (I):

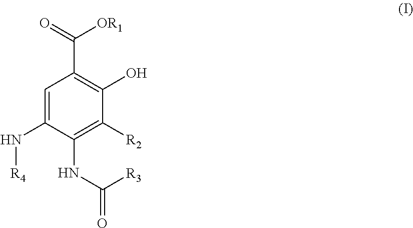

wherein R₁ group is H, CH₃, or C₂H₅; R₂ group is H, or Br; R₃ group is CH₃, or C₃H₇; and R₄ group is H, or C(═NH)—NH₂.

Preferably, totally 11 synthesized compounds were obtained: 4-(amido)-5-amino-2-hydroxybenzoic acid (compounds 6a, 6c), alkyl 4-(amido)-5-amino-2-hydroxybenzoate, (compounds 6b, 6d, 6e), methyl 4-(acetamido)-5-amino-3-bromo-2-hydroxybenzoate (compound 6f), 4-(acetamido)-5-guanidino-2-hydroxybenzoic acid (compound 7a), alkyl 4-(amido)-5-guanidino-2-hydroxybenzoate (compounds 7b, 7e), methyl 4-(acetamido)-3-bromo-5-guanidino-2-hydroxybenzoate (compound 7f), and 4-(acetamido)-5-amino-3-bromo-2-hydroxybenzoic acid (compound 8).

Preferably, totally 11 intermediates were obtained: methyl 4-amino-2-hydroxybenzoate (compound 1), 4-(amido)-2-hydroxybenzoic acid (compounds 2a, 2c), methyl 4-(amido)-2-hydroxybenzoate (compounds 2b, 2d), 4-(amido)-2-hydroxyl-5-nitrobenzoic acid (compounds 3a, 3c), alkyl 4-(amido)-2-hydroxyl-5-nitrobenzoate (compounds 3b, 3d, 4), methyl 4-(acetamido)-3-bromo-2-hydroxyl-5-nitrobenzoate (compound 5).

In another aspect, the present invention relates to an anti-influenza pharmaceutical composition, comprising the 4,5-diamino-3-halo-2-hydroxybenzoic acid derivatives or the pharmaceutically acceptable salts thereof.

In yet another aspect, the present invention relates to a method of treating influenza caused by a virus, comprising administrating to a subject suffering from influenza with an effective amount of the 4,5-diamino-3-halo-2-hydroxybenzoic acid derivatives.

Preferably, the 4,5-diamino-3-halo-2-hydroxybenzoic acid derivative is adapted to inhibit influenza neuraminidase of the subject.

Preferably, the influenza is type A influenza or type B influenza.

Preferably, the virus is influenza virus type A subtype H1N1.

In yet another aspect, the present invention relates to a method of preparing the 4,5-diamino-3-halo-2-hydroxybenzoic acid derivatives, comprising the steps of: providing p-aminosalicylic acid (PAS) as a initial reagent, wherein the carboxyl group on C-1 site is kept or converted to an ester group; the hydrogen atom on C-3 site is kept or converted to a bromo group, the amino group on C-4 site is converted to a amido group, the hydrogen atom on C-5 site is converted to amino or guanidino group;

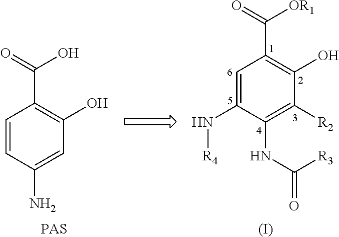

wherein R₁ group is H, CH₃, or C₂H₅; R₂ group is H, or Br; R₃ group is CH₃, or C₃H₇; and R₄ group is H, or C(═NH)—NH₂.

Preferably, the 4,5-diamino-3-halo-2-hydroxybenzoic acid derivative is methyl 4-(acetamido)-5-amino-3-bromo-2-hydroxybenzoate (compound 6f).

Preferably, the method further comprising a step of adding the methyl 4-(acetamido)-5-amino-3-bromo-2-hydroxybenzoate (compound 6f) to NaOH solution to form 4-(acetamido)-5-amino-3-bromo-2-hydroxybenzoic acid (compound 8). 4,5-diamino-3-halo-2-hydroxybenzoic acid derivatives provided here were non-toxic to MDCK cells, particularly compounds 6a, 6b, 6c, 6e, 6f, 7a, 7b and 8 had better anti-H1N1 activity. In the future, these compounds can be used to focus on viral neuraminidases as targets to develop effective anti-influenza drugs.

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### BRIEF DESCRIPTION OF THE DRAWINGS

The related drawings in connection with the detailed description of the present invention to be made later are described briefly as follows, in which:

FIG. 1 demonstrates the structure of influenza virus;

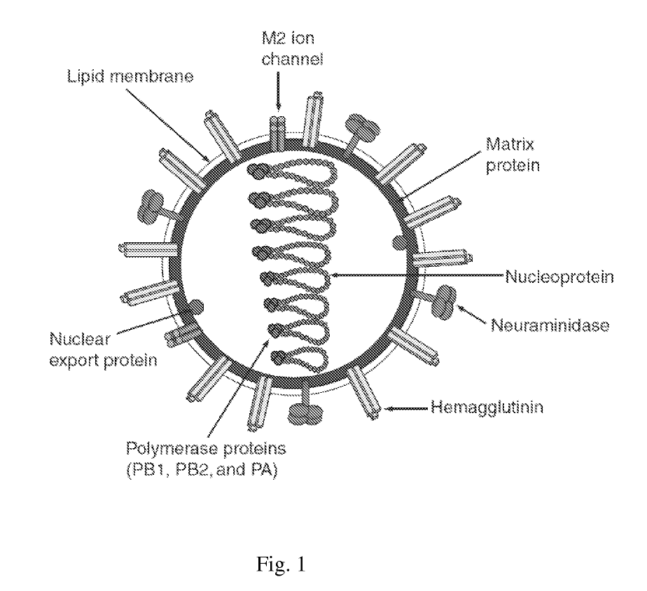

FIG. 2 demonstrates the structure of hemagglutinin monomer and its conformational change;

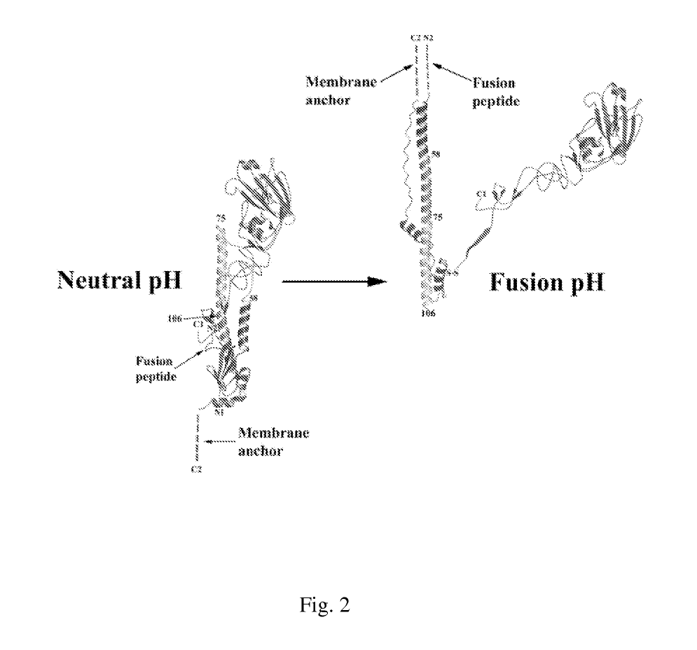

FIG. 3 demonstrates the structure of neuraminidase tetramer and its active site bound with oseltamivir;

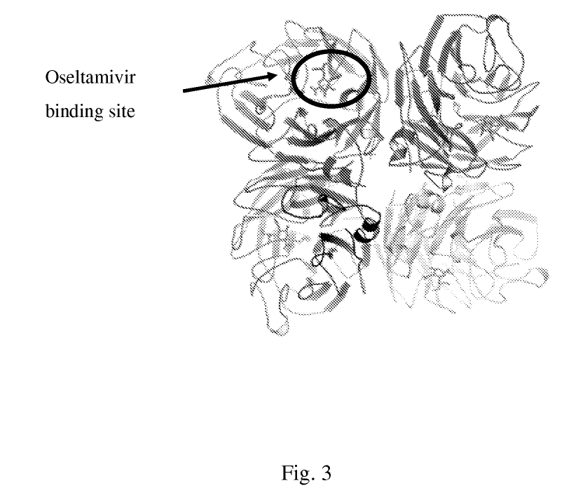

FIG. 4 demonstrates the interaction of DANA inhibitor and the active site of neuraminidase; and

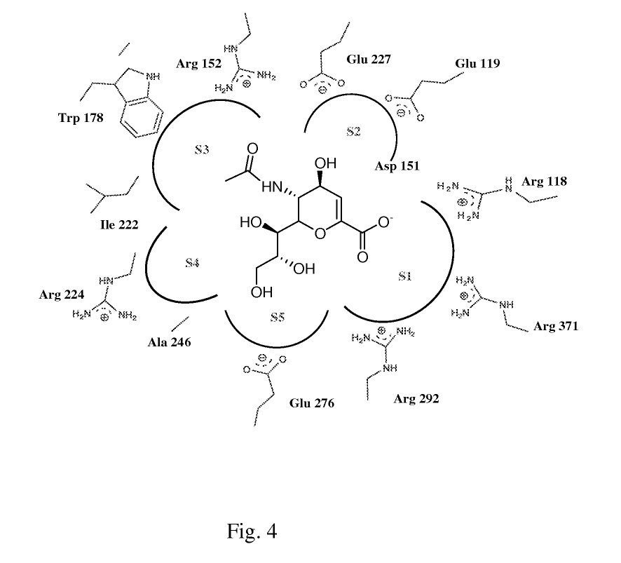

FIG. 5 demonstrates the cell culture process of anti-influenza assay.

### DETAILED DESCRIPTION OF THE INVENTION

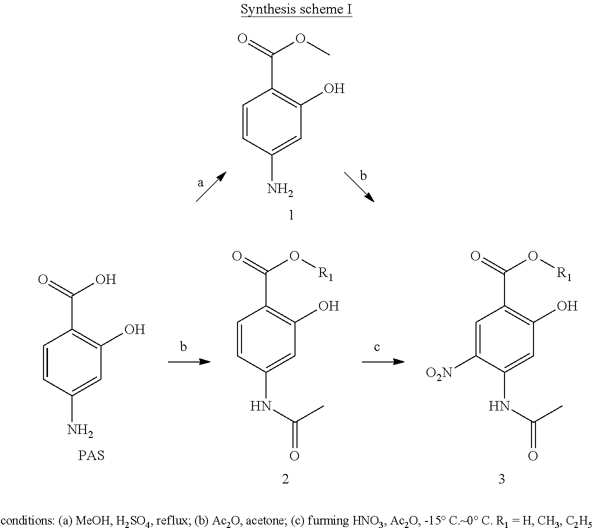

As shown in synthesis scheme I: methyl 4-amino-2-hydroxybenzoate (compound 1) was prepared by refluxing p-aminosalicylic (PAS) acid and methanol in the presence of concentrated sulfuric acid (esterification). PAS and compound 1 were respectively reacted with acetic anhydride in dry acetone to form their acetamido derivatives (compound 2). The compound 2 were then reacted with fuming nitric acid in acetic anhydride to form nitro derivatives (compound 3).

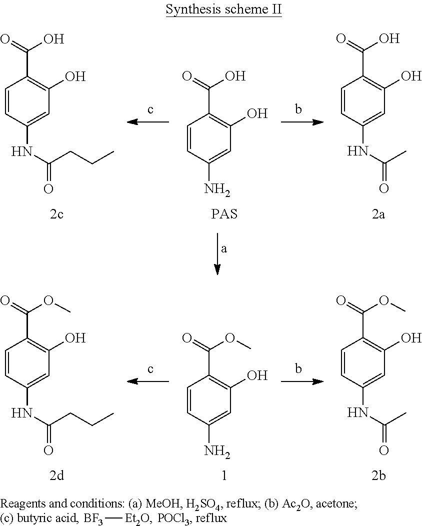

As shown in synthesis scheme II: methyl 4-amino-2-hydroxybenzoate (compound 1) was prepared by the esterification reaction of p-aminosalicylic (PAS) acid and methanol in the presence of concentrated sulfuric acid. PAS and compound 1 were respectively reacted with acetic anhydride in dry acetone to form their acetamido derivatives, 4-acetamido-2-hydroxybenzoic acid (compound 2a) and methyl 4-acetamido-2-hydroxybenzoate (compound 2b). Additionally, PAS and compound 1 were respectively reacted with butyric acid in boron trifluororide etherate and phosphorus oxychloride to form 4-(butyramido)-2-hydroxybenzoic acid (compound 2c) and methyl 4-(butyramido)-2-hydroxybenzoate (compound 2d).

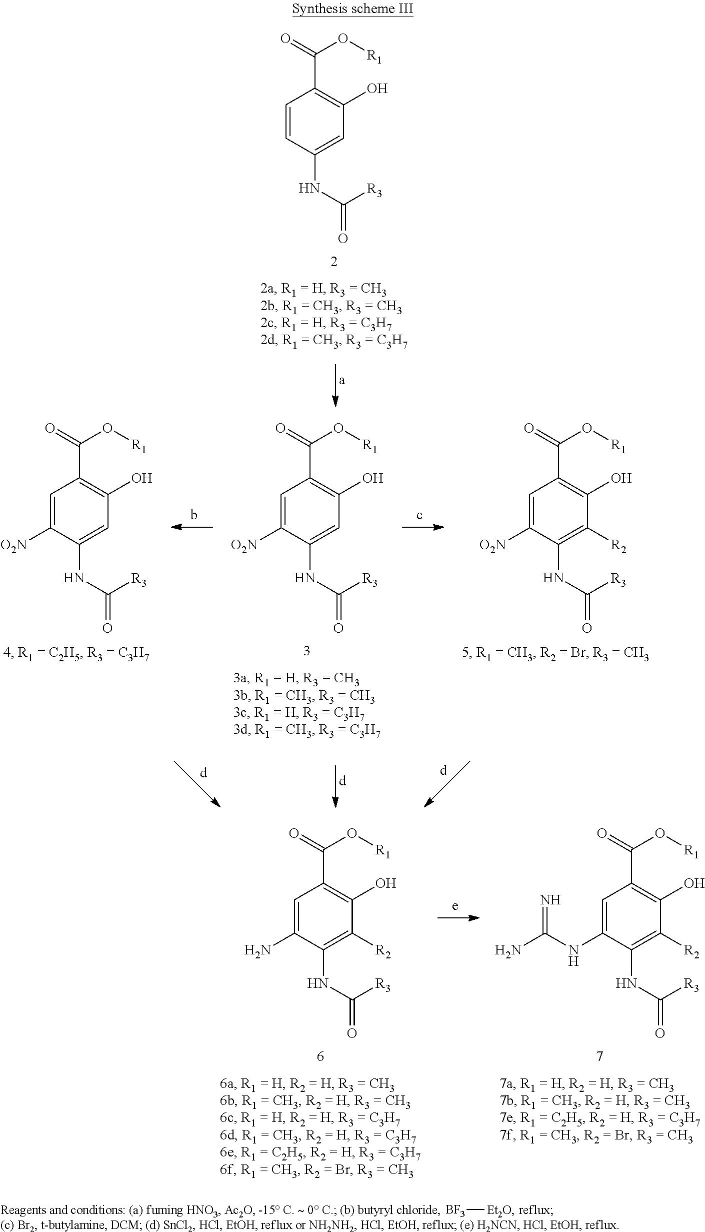

A series of PAS derivatives (compound 2) were reacted with fuming nitric acid to form their nitro derivatives (compound 3). Methyl 4-acetamido-2-hydroxy-5-nitrobenzoate (compound 3b) was reacted with butyryl chloride in boron trifluororide etherate to form ethyl 4-(butyramido)-2-hydroxy-5-nitrobenzoate (compound 4). 4-acetamido-2-hydroxy-5-nitrobenzoic acid was reacted with bromine to form methyl 4-acetamido-3-bromo-2-hydroxy-nitrobenzoate (compound 5). A series of nitro derivatives (compounds 3, 4 and 5) were hydrogenated with tin chloride or hydrazine to form another series of nitro derivatives (compound 6), and the nitro derivatives (compound 6) were then reacted with cyanamide to form a series of guanidine derivatives (compound 7).

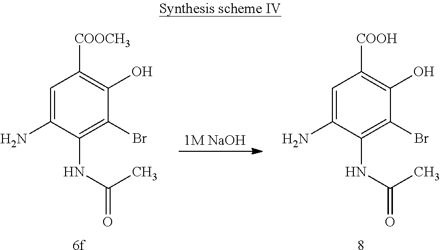

Compound 6f hydrolyzed in 1N NaOH solution to form a carboxylic acid derivative (compound 8).

The synthesized products of the present invention have been identified by their physical characteristics, absorption spectrum, mass spectrum and NMR spectrum. The synthesized products were totally 11 as follows: 4-(amido)-5-amino-2-hydroxybenzoic acid (compounds 6a, 6c), alkyl 4-(amido)-5-amino-2-hydroxybenzoate, (compounds 6b, 6d, 6e), methyl 4-(acetamido)-5-amino-3-bromo-2-hydroxybenzoate (compound 6f), 4-(acetamido)-5-guanidino-2-hydroxybenzoic acid (compound 7a), alkyl 4-(amido)-5-guanidino-2-hydroxybenzoate (compounds 7b, 7e), methyl 4-(acetamido)-3-bromo-5-guanidino-2-hydroxybenzoate (compound 7f), and 4-(acetamido)-5-amino-3-bromo-2-hydroxybenzoic acid (compound 8).

There were totally 11 synthesized intermediates as follows: methyl 4-amino-2-hydroxybenzoate (compound 1), (4-(amido)-2-hydroxybenzoic acid (compounds 2a, 2c), methyl 4-(amido)-2-hydroxybenzoate (compounds 2b, 2d), 4-(amido)-2-hydroxyl-5-nitrobenzoic acid (compounds 3a, 3c), alkyl 4-(amido)-2-hydroxyl-5-nitrobenzoate (compounds 3b, 3d, 4), methyl 4-(acetamido)-3-bromo-2-hydroxyl-5-nitrobenzoate (compound 5).

The methods and materials of following embodiments were performed according to the contents as previously described.

### Example 1

### Synthesis of methyl 4-amino-2-hydroxybenzoate (compound 1)

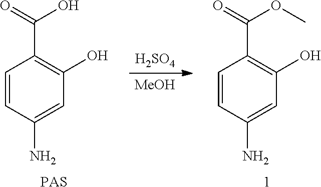

To a solution of p-aminosalicyclic acid (15.3 g, 100.1 mmol) in anhydrous methanol (230 ml) was added concentrated H₂SO₄ (15 ml) slowly in an ice bath with magnetic stirring. The reaction mixture was refluxed for 48 h. After cooling to room temperature, the solvent was evaporated in vacuo. The resulting residue was basified with saturated NaHCO₃ and extracted with ethyl acetate. The organic layer was then acidified with dilute HCl and extracted with water. The organic layer was dried over Na₂SO₄, filtered and concentrated to provide compound 1 (13.8 g, 86%) as brown powders.

Compound 1: Mwt: 167.16; R_{f}: 0.47 (ethyl acetate:hexane=1:2); ¹H-NMR (DMSO-d₆, 300 MHz) δ (ppm): 3.77 (3H, s, OCH₃), 5.98 (1H, d, J=2.1 Hz, Ar H-3), 6.10 (1H, dd, J=6.6, 2.1 Hz, Ar H-5), 6.14 (2H, s, NH₂), 7.43 (1H, d, J=8.7 Hz, Ar H-6), 10.76 (1H, s, OH).

### Example 2

### Synthesis of 4-(acetamido)-2-hydroxybenzoic acid (compound 2a)

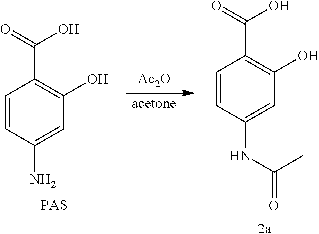

To a solution of p-aminosalicyclic acid (15.3 g, 100.1 mmol) in anhydrous acetone (130 ml) was added acetic anhydride (10 ml, 105.8 mmol) in an ice bath with magnetic stirring. After being stirred for 24 h, the solvent was evaporated in vacuo, and the solid residue was washed with water and filtered to provide compound 2a (18.5 g, 95%) as white powders.

Compound 2a: Mwt: 195.17; R_{f}: 0.31 (dichloromethane:methanol=3:1). ¹H-NMR (DMSO-d₆, 300 MHz) δ (ppm): 2.05 (3H, s, CH₃), 7.02 (1H, dd, J=6.9, 2.0 Hz, Ar H-5), 7.33 (1H, d, J=2.1 Hz, Ar H-3), 7.68 (1H, d, J=8.7 Hz, Ar H-6), 10.18 (1H, s, ArNH), 11.34 (1H, s, OH).

### Example 3

### Synthesis of methyl 4-(acetamido)-2-hydroxybenzoate (compound 2b)

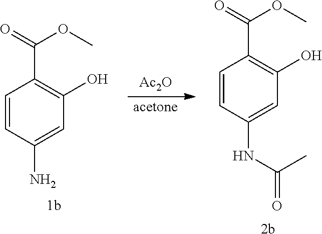

To a solution of compound 1b (2.46 g, 15.3 mmol) in anhydrous acetone (25 ml) was added acetic anhydride (3 ml, 31.7 mmol) in an ice bath with magnetic stirring. After being stirred for 19 h, the solvent was evaporated in vacuo, and the solid residue was washed with water and filtered to provide compound 2b (2.74 g, 86%) as white powders.

Compound 2b: Mwt: 209.20; R_{f}: 0.50 (ethyl acetate:hexane=1:1). ¹H-NMR (DMSO-d₆, 300 MHz) δ (ppm): 2.06 (3H, s, CH₃), 3.85 (3H, s, OCH₃), 7.04 (1H, dd, J=6.6, 2.1 Hz, Ar H-5), 7.37 (1H, d, J=1.8 Hz, Ar H-3), 7.70 (1H, d, J=8.7 Hz, Ar H-6), 10.22 (1H, s, ArNH), 10.6 (1H, s, OH).

### Example 4

### Synthesis of 4-(butyramido)-2-hydroxybenzoic acid (compound 2c)

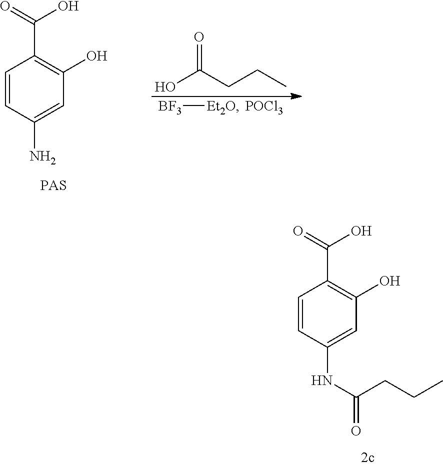

To a mixture of p-aminosalicyclic acid (3 g, 20 mmol) and butyric acid (5 ml, 55 mmol) in POCl₃ (30 ml) was added BF₃-Et₂O (7 ml) in an ice bath with magnetic stirring. The reaction mixture was then refluxed for 0.5 hr. After cooling to room temperature, the reaction mixture was poured into cold dilute HCl slowly. The precipitate was filtered with cold water to give crude product. The solid was then washed with ether to provide compound 2c (1.17 g, 26%) as pale yellow powders.

Compound 2c: Mwt: 223.23; R_{f}: 0.25 (ethyl acetate:hexane=2:1). ¹H-NMR (DMSO-d₆, 300 MHz) δ (ppm): 0.89 (3H, t, J=7.5 Hz, CH₂CH₂CH₃), 1.59 (2H, m, CH₂CH₂CH₃), 2.29 (2H, t, J=7.2, CH₂CH₂CH₃), 7.04 (1H, dd, J=6.6, 2.0 Hz, Ar H-5), 7.35 (1H, d, J=2.1 Hz, Ar H-3), 7.68 (1H, d, J=8.7 Hz, Ar H-6), 10.13 (1H, s, ArNH), 11.30 (1H, s, OH).

### Example 5

### Synthesis of methyl 4-(butyramido)-2-hydroxybenzoate (compound 2d)

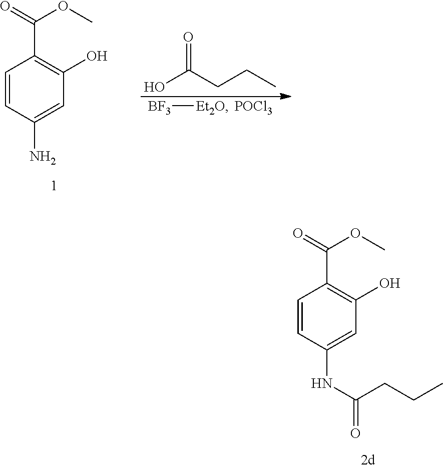

To a mixture of compound 1b (1 g, 6.2 mmol) and butyric acid (1.5 ml, 14.4 mmol) in POCl₃ (8 ml) was added BF₃-Et₂O (4 ml) in an ice bath with magnetic stiffing. The reaction mixture was then refluxed for 20 min. After cooling to room temperature, the reaction mixture was poured into cold dilute HCl slowly. The precipitate was filtered and washed with cold water to provide compound 2d (0.77 g, 52%) as pale yellow powders.

Compound 2d: Mwt: 237.25; R_{f}: 0.47 (ethyl acetate:hexane=1:7). ¹H-NMR (DMSO-d₆, 300 MHz) δ (ppm): 0.89 (3H, t, J=7.4 Hz, CH₂CH₂CH₃), 1.59 (2H, m, CH₂CH₂CH₃), 2.30 (2H, t, J=7.2 Hz, CH₂CH₂CH₃), 3.85 (3H, s, OCH₃), 7.07 (1H, dd, J=6.9, 2.1 Hz, Ar H-5), 7.39 (1H, d, J=2.1 Hz, Ar H-3), 7.70 (1H, d, J=8.7 Hz, Ar H-6), 10.16 (1H, s, ArNH), 10.60 (1H, s, OH).

### Example 6

### Synthesis of 4-(amido)-2-hydroxyl-5-nitrobenzoic acid (compounds 3a, 3c)

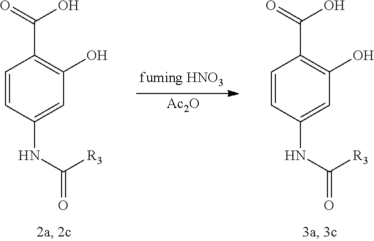

To a solution of compound 2a or 2c (2.7 mmol) in acetic anhydride (ml) was added fuming nitric acid slowly (0.13 ml, 2.9 mmol) at −20° C. with magnetic stirring. The reaction mixture was stirred at −20˜−15° C. for 24 hr, and then warmed to 0° C. until the reaction was complete as evidenced by TLC. The reaction mixture was poured into cold water and the precipitate was filtered with cold water to give crude product. The solid was washed with ethanol and then the collected filtrate was concentrated and washed with acetone to provide compound 3a or 3c (as shown in table 1).

TABLE 1

| | | 2a, 2c | Yield | |
|---|---|---|---|---|
| compound | R₃ | (g) | g (%) | appearance |
| 3a | —CH₃ | 0.52 | 0.15 (23%) | yellow |
| 3c | —C₃H₇ | 0.60 | 0.22 (30%) | yellow |

Compound 3a: Mwt: 240.17; R_{f}: 0.25 (dichloromethane:methanol=4:1). ¹H-NMR (DMSO-d₆, 300 MHz) δ (ppm): 2.14 (3H, s, CH₃), 7.54 (1H, s, Ar H-3), 8.46 (1H, s, Ar H-6), 10.44 (1H, s, ArNH).

Compound 3c: Mwt: 268.22; R_{f}: 0.3 (dichloromethane:methanol=4:1). ¹H-NMR (DMSO-d₆, 300 MHz) δ (ppm): 0.92 (3H, t, J=7.4 Hz, CH₂CH₂CH₃), 1.61 (2H, m, CH₂CH₂CH₃), 2.40 (2H, t, J=7.2, CH₂CH₂CH₃), 7.65 (1H, s, Ar H-3), 8.45 (1H, s, Ar H-6), 10.41 (1H, s, ArNH).

### Example 7

### Synthesis of methyl 4-(amido)-2-hydroxyl-5-nitrobenzoate (compounds 3b, 3d)

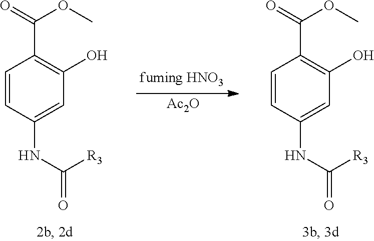

To a solution of compound 2b or 2d (1.9 mmol) in acetic anhydride (5 ml) was added fuming nitric acid (0.1 ml, 2.2 mmol) slowly at −20° C. with magnetic stirring. The reaction mixture was stirred at −5° C. for 15 min. The reaction mixture was poured into cold water and the precipitate was filtered with cold water to give crude product. The solid was then washed with ethanol to provide compound 3b or 3d.

TABLE 2

| | | 2b, 2d | Yield | |
|---|---|---|---|---|
| compound | R₃ | (g) | g (%) | appearance |
| 3b | —CH₃ | 0.40 | 0.19 (39%) | yellow |
| 3d | —C₃H₇ | 0.60 | 0.22 (36%) | yellow |

Compound 3b: Mwt: 254.20; R_{f}: 0.52 (ethyl acetate:hexane=1:7). ¹H-NMR (DMSO-d₆, 300 MHz) δ (ppm): 2.16 (3H, s, CH₃), 3.86 (3H, s, OCH₃), 7.56 (1H, s, Ar H-3), 8.44 (1H, s, Ar H-6), 10.41 (1H, s, ArNH), 11.40 (1H, s, OH).

Compound 3d: Mwt: 282.25; R_{f}: 0.66 (ethyl acetate:hexane=1:7). ¹H-NMR (DMSO-d₆, 300 MHz) δ (ppm): 0.92 (3H, t, J=7.4 Hz, CH₂CH₂CH₃), 1.61 (2H, m, CH₂CH₂CH₃), 2.41 (2H, t, J=7.2, CH₂CH₂CH₃), 3.85 (3H, s, OCH₃), 7.76 (1H, s, Ar H-3), 8.45 (1H, s, Ar H-6), 10.40 (1H, s, ArNH), 11.42 (1H, s, OH).

### Example 8

### Synthesis of ethyl 4-(butyramido)-2-hydroxyl-5-nitrobenzoate (compound 4)

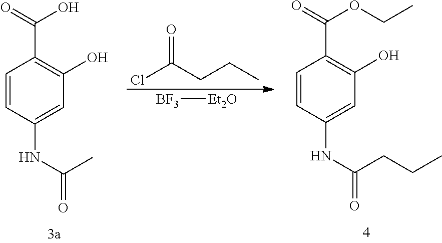

A mixture of compound 3a (1 g, 4.2 mmol) and butyric acid (1.5 ml, 14.4 mmol) in BF₃-Et₂O (8 ml) was refluxed for 1.5 hr. After cooling to room temperature, the reaction mixture was poured into cold water slowly. The precipitate was filtered and washed with cold water. The residue was then purified by column chromatography on silica gel with n-hexane/ethyl acetate (8:1) to provide compound 4 (0.2 g, 16%) as brown powders.

Compound 4: Mwt: 296.28; R_{f}: 0.47 (ethyl acetate:hexane=1:9). ¹H-NMR (DMSO-d₆, 300 MHz) δ (ppm): 0.92 (3H, t, J=7.5 Hz, CH₂CH₂CH₃), 1.32 (3H, t, J=7.1 Hz, COCH₂CH₃), 1.61 (2H, m, CH₂CH₂CH₃), 2.41 (2H, t, J=7.2 Hz, CH₂CH₂CH₃), 4.33 (2H, q, J=7.2 Hz, COCH₂CH₃), 7.74 (1H, s, Ar H-3), 8.44 (1H, s, Ar H-6), 10.41 (1H, s, NH), 11.42 (1H, s, OH).

### Example 9

### Synthesis of methyl 4-(acetamido)-3-bromo-2-hydroxyl-5-nitrobenzoate (compound 5)

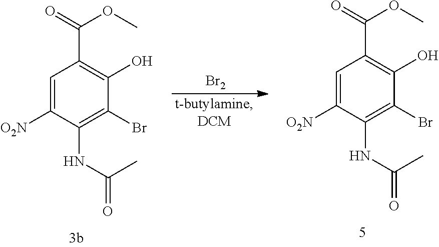

Br₂ (0.2 ml, 3.9 mmol) was added to a solution of compound 3b (0.8 g, 3.3 mmol) and t-butylamine (0.37 ml, 3.5 mmol) in chloroform (10 ml) at 0° C. After being stirred for 1 hr at room temperature, the reaction mixture was treated with Na₂S₂O₃ solution, followed by basified with saturated NaHCO₃ solution. Chloroform was then evaporated in vacuo. The residue was treated with Na₂S₂O₃ solution, acidified with hydrochloric acid and extracted with ethyl acetate. The organic layer was dried over Na₂SO₄, filtered and concentrated to provide compound 5 (0.84 g, 88%) as pearl powders.

Compound 5: Mwt: 333.09; R_{f}: 0.25 (ethyl acetate:hexane=2:1). ¹H-NMR (DMSO-d₆, 300 MHz) δ (ppm): 2.07 (3H, s, CH₃), 3.95 (3H, s, OCH₃), 8.43 (1H, s, Ar H-6), 10.38 (1H, s, ArNH), 11.80 (1H, s, OH).

### Example 10

### Synthesis of 4-(amido)-5-amino-2-hydroxybenzoic acid (compounds 6a, 6c)

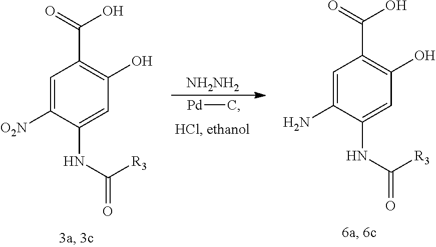

To a suspension of compound 3a or 3c (2.0 mmol) in EtOH (10 mL) was added catalytic quantity of 10% Pd—C and 5% HCl (1 mL). Hydrazine hydrate (80%, 0.35 mL) dissolved in EtOH (10 mL) was then added slowly to the above mixture in ice bath. The reaction mixture was then refluxed for 24 hr. The Pd—C was filtered through Celite and the ethanol was concentrated under vacuum to give crude product. The residue obtained was recrystallized from MeOH to give compound 6a or 6c.

TABLE 3

| | | 3a, 3c | Yield | |
|---|---|---|---|---|
| compound | R₃ | (g) | g (%) | appearance |
| 6a | —CH₃ | 0.46 | 0.23 (56%) | brown |
| 6c | —C₃H₇ | 0.53 | 0.14 (29%) | brown |

Compound 6a: Mwt: 210.19; R_{f}: 0.50 (ethyl acetate:methanol=2:1); mp: 295-297° C.; UV (MeOH): λₘₐₓ nm (log ε)=219 (1.43). HRMS (EI) m/z (%): calcd.: 210.0641 (M⁺). found: 192.0529 (M-18, 5), 148.0633 (100). ¹H-NMR (D₂O/CD₃COOD, 300 MHz) δ (ppm): 1.30 (3H, s, CH₃), 5.66 (1H, s, Ar H-6), 6.73 (1H, s, Ar H-3). ¹³C-NMR (DMSO-d₆, 75 MHz) δ (ppm): 12.34, 99.59, 111.77, 115.59, 124.54, 136.10, 153.54, 158.61, 171.39.

Compound 6c: Mwt: 238.24; R_{f}: 0.26 (ethyl acetate:methanol=6:1); mp: 198-200° C.; UV (MeOH): λₘₐₓ nm (log ε)=219 (1.35). HRMS (EI) m/z (%): calcd.: 238.0954 (M⁺). found: 220.0893 (M-18, 32.2), 202.0736 (100). ¹H-NMR (DMSO-d₆, 300 MHz) δ (ppm): 0.93 (3H, t, J=7.4 Hz, CH₂CH₂CH₃), 1.76 (2H, m, CH₂CH₂CH₃), 2.76 (2H, t, J=7.1 Hz, CH₂CH₂CH₃), 6.81 (1H, s, Ar H-6), 7.20 (1H, s, NH₂), 7.89 (1H, s, Ar H-3). ¹³C-NMR (DMSO-d₆, 75 MHz) δ (ppm): 13.34, 21.07, 30.08, 98.67, 110.14, 116.49, 156.76, 157.83, 172.41.

### Example 11

### Synthesis of alkyl 4-(amido)-5-amino-2-hydroxybenzoate (compounds 6b, 6d, 6e)

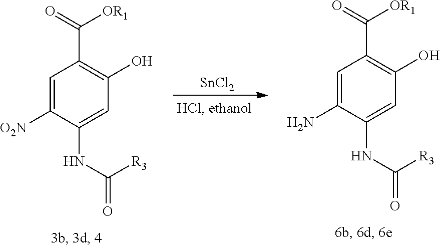

To a mixture of compound 3b, 3d or 4 (5.9 mmol) and SnCl₂.2H₂O (6 g, 56.3 mmol) in ethanol (40 ml) was added 2 drops of concentrated HCl with magnetic stiffing. The reaction mixture was then refluxed for 1˜1.5 hr. After cooling to room temperature, the solvent was evaporated in vacuo. The resulting residue was poured into water, basified with saturated NaHCO₃ solution, and filtered with water and ethyl acetate. The collected filtrate was extracted with ethyl acetate and water. The organic layer was then dried over Na₂SO₄, filtered and concentrated to provide compound 6b, 6d or 6e.

TABLE 4

| | | | 3b, 3d, 4 | Yield | |
|---|---|---|---|---|---|
| compound | R₁ | R₃ | (g) | g (%) | appearance |
| 6b | —CH₃ | —CH₃ | 1.5 | 0.73 (55%) | pale yellow |
| 6d | —CH₃ | —C₃H₇ | 0.4 | 0.12 (30%) | pale yellow |
| 6e | —C₂H₅ | —C₃H₇ | 1.74 | 0.49 (32%) | white |

Compound 6b: Mwt: 224.21; R_{f}: 0.47 (ethyl acetate); mp: 204-206° C.; UV (MeOH): λₘₐₓ nm (log ε)=225 (2.37). HRMS (EI) m/z (%): calcd.: 224.0797 (M⁺). found: 206.0688 (M-18, 22.6), 174.0426 (100). ¹H-NMR (DMSO-d₆, 300 MHz) δ (ppm): 2.40 (3H, s, CH₃), 3.90 (3H, s, OCH₃), 6.90 (1H, s, Ar H-6), 7.89 (1H, s, Ar H-3), 10.53 (1H, s, ArNH), 12.26 (1H, s, OH). ¹³C-NMR (DMSO-d₆, 75 MHz) δ (ppm): 14.51, 52.22, 97.01, 106.73, 119.03, 137.21, 140.58, 153.68, 156.53, 170.70.

Compound 6d: Mwt: 252.27; R_{f}: 0.33 (ethyl acetate:hexane=1:1); mp: 132-134° C.; UV (MeOH): λₘₐₓ nm (log ε)=233 (3.42). ¹H-NMR (DMSO-d₆, 300 MHz) δ (ppm): 0.93 (3H, t, J=7.2 Hz, CH₂CH₂CH₃), 1.70˜1.82 (2H, m, CH₂CH₂CH₃), 2.74 (2H, t, J=7.7 Hz, CH₂CH₂CH₃), 3.90 (3H, s, OCH₃), 6.91 (1H, s, Ar H-6), 7.90 (1H, s, Ar H-3), 10.53 (1H, s, ArNH), 12.22 (1H, s, OH). ¹³C-NMR (DMSO-d₆, 75 MHz) δ (ppm): 13.34, 20.38, 30.30, 52.10, 99.28, 106.74, 116.49, 134.05, 143.50, 156.20, 158.25, 170.54.

Compound 6e: Mwt: 266.29; R_{f}: 0.44 (ethyl acetate:hexane=2:1); mp: 135-137° C.; UV (MeOH): λₘₐₓ nm (log ε)=202.5 (0.31). HRMS (EI) m/z (%): calcd.: 266.1267 (M⁺) found: 248.1168 (M-18, 20.2), 202.0740 (100). ¹H-NMR (DMSO-d₆, 300 MHz) δ (ppm): 0.90 (3H, t, J=7.5 Hz, CH₂CH₂CH₃), 1.33 (3H, t, J=7.1 Hz, COCH₂CH₃), 1.67˜1.79 (2H, m, CH₂CH₂CH₃), 2.72 (2H, t, J=7.5 Hz, CH₂CH₂CH₃), 4.34 (2H, q, J=7.2 Hz, OCH₂CH₃), 6.88 (1H, s, Ar H-6), 7.90 (1H, s, Ar H-3), 10.62 (1H, s, ArNH), 12.23 (1H, s, OH). ¹³C-NMR (DMSO-d₆, 75 MHz) δ (ppm): 13.75, 14.25, 20.85, 30.57, 61.57, 99.54, 107.49, 117.66, 156.59, 158.88, 170.59.

### Example 12

### Synthesis of methyl 4-(acetamido)-5-amino-3-bromo-2-hydroxybenzoate (compound 6f)

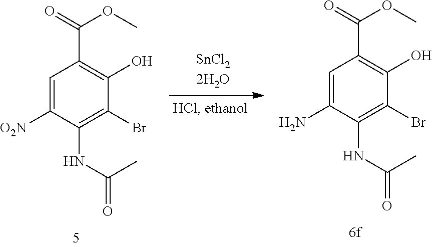

To a mixture of compound 5 (0.84 g, 2.9 mmol) and SnCl₂·2H₂O (3.2 g, 30 mmol) in ethanol (20 ml) was added 2 drops of concentrated HCl with magnetic stiffing. The reaction mixture was then refluxed for 1 hr. After cooling to room temperature, the solvent was evaporated in vacuo. The resulting residue was poured into water, basified with saturated NaHCO₃ solution, and filtered with water and ethyl acetate. The collected filtrate was extracted with ethyl acetate and water. The organic layer was then dried over Na₂SO₄, filtered and concentrated to provide compound 6f (0.13 g, 17%) as pale yellow powders.

Compound 6f: Mwt: 303.11; R_{f}: 0.40 (ethyl acetate); mp: 217-219° C.; UV (MeOH): λₘₐₓ nm (log ε)=228.5 (1.94). HRMS (EI) m/z (%): calcd.: 301.9902 (M⁺). found: 283.9811 (M-18, 5.3), 253.9510 (100). ¹H-NMR (DMSO-d₆, 300 MHz) δ (ppm): 2.52 (3H, s, CH₃), 3.95 (3H, s, OCH₃), 7.83 (1H, s, Ar H-6), 11.04 (1H, s, ArNH), 12.26 (1H, s, OH). ¹³C-NMR (DMSO-d₆, 75 MHz) δ (ppm): 12.06, 52.77, 98.33, 108.06, 108.97, 126.21, 141.89, 152.40, 153.49, 170.04.

### Example 13

### Synthesis of 4-(acetamido)-5-guanidino-2-hydroxybenzoic acid (compound 7a)

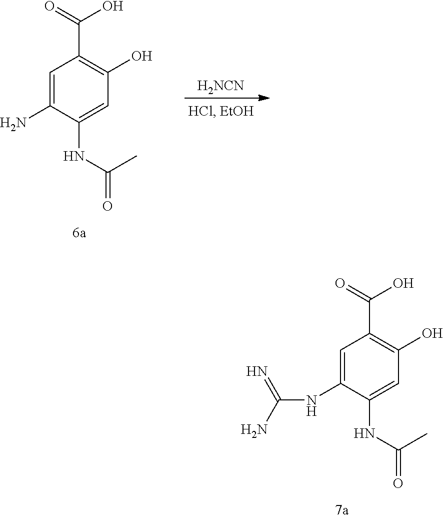

To a mixture of compound 6a (0.63 g, 3 mmol), cyanamide (1.26 g, 30 mmol) in ethanol (40 ml) was added 2 drops of concentrated HCl with magnetic stiffing. The reaction mixture was then refluxed for 24 hr. After cooling to room temperature, the solvent was evaporated in vacuo. The resulting solid was washed with ethanol to provide compound 7a (0.42 g, 56%) as brown powders.

Compound 7a: Mwt: 252.23; R_{f}: 0.44 (ethyl acetate:methanol=4:1); mp: 334-357° C.; UV (MeOH): λₘₐₓ nm (log ε)=217.5 (2.49). HRMS (EI) m/z (%): calcd.: 252.0859 (M⁺). found: 192.0529 (M-60, 2.5), 174.0426 (100). ¹H-NMR (DMSO-d₆, 300 MHz) δ (ppm): 2.52 (3H, s, CH₃), 7.41 (1H, s, Ar H-6), 7.96 (1H, s, Ar H-3), 11.90 (1H, s, OH).

### Example 14

### Synthesis of methyl 4-(acetamido)-5-guanidino-2-hydroxybenzoate (compound 7b)

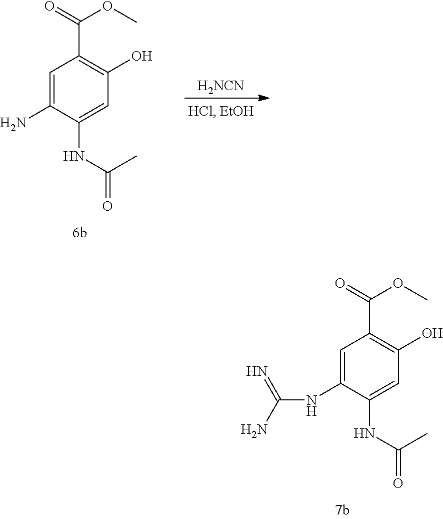

To a mixture of compound 6b (0.12 g, 0.54 mmol), cyanamide (0.22 g, 5.2 mmol) in ethanol (10 ml) was added 2 drops of concentrated HCl with magnetic stiffing. The reaction mixture was then refluxed for 24 hr. After cooling to room temperature, the solvent was evaporated in vacuo. The resulting solid was washed with ethyl acetate to provide compound 7b (0.13 g, 93%) as white powders.

Compound 7b: Mwt: 266.25; R_{f}: 0.40 (ethyl acetate:hexane=4:1); mp 233-234° C.; UV (MeOH): λₘₐₓ nm (log ε)=225 (2.75). HRMS (EI) m/z (%): calcd.: 266.1015 (M⁺). found: 206.0648 (M-60, 100). ¹H-NMR (DMSO-d₆, 300 MHz) δ (ppm): 2.45 (3H, s, CH₃), 3.90 (3H, s, OCH₃), 5.41 (4H, s, NHCNH(NH₂)), 6.91 (1H, s, Ar H-6), 7.89 (1H, s, Ar H-3), 8.42 (1H, s, OH), 10.54 (1H, s, ArNHCO). ¹³C-NMR (DMSO-d₆, 75 MHz) δ (ppm): 14.51, 52.24, 99.33, 106.81, 116.38, 134.16, 143.54, 154.73, 156.29, 159.63, 170.63.

### Example 15

### Synthesis of ethyl 4-(butyramido)-5-guanidino-2-hydroxybenzoate (compound 7e)

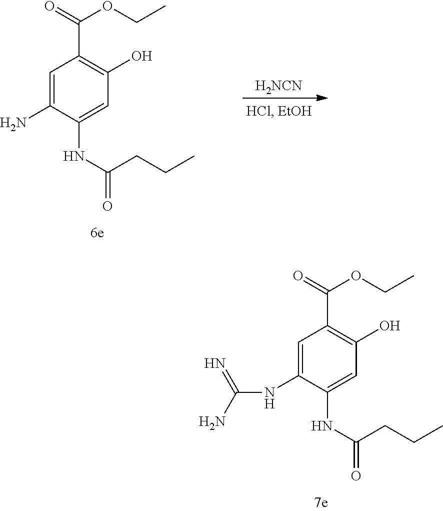

To a mixture of compound 6e (0.13 g, 0.48 mmol), cyanamide (0.4 g, 9.6 mmol) in ethanol (10 ml) was added 2 drops of concentrated HCl with magnetic stiffing. The reaction mixture was then refluxed for 17 hr. After cooling to room temperature, the solvent was evaporated in vacuo and extracted with ethyl acetate and NaHCO₃ solution. The organic layer was then dried over Na₂SO₄, filtered and concentrated to provide compound 7e (0.02 g, 13%) as white powders.

Compound 7e: Mwt: 308.33; R_{f}: 0.47 (ethyl acetate:hexane=3:1); UV (MeOH): λₘₐₓ nm (log ε)=216.5 (2.02). HRMS (EI) m/z (%): calcd.: 308.1485 (M⁺). found: 248.1154 (M-60, 28.7), 202.0741 (100). ¹H-NMR (CD₃OD, 300 MHz) δ (ppm): 1.01 (3H, t, J=7.4 Hz, CH₂CH₂CH₃), 1.43 (3H, t, J=7.1 Hz, COCH₂CH₃), 1.76˜1.91 (2H, m, CH₂CH₂CH₃), 2.84 (2H, t, J=7.5 Hz, CH₂CH₂CH₃), 4.44 (2H, q, J=7.1 Hz, COCH₂CH₃), 4.61 (4H, s, NHCNH(NH₂)), 6.93 (1H, s, Ar H-6), 8.05 (1H, s, Ar H-3).

### Example 16

### Synthesis of methyl 4-(acetamido)-3-bromo-5-guanidino-2-hydroxybenzoate (compound 7f)

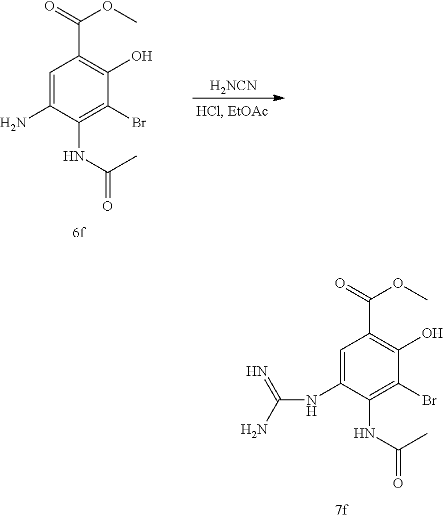

To a mixture of compound 6f (0.081 g, 0.27 mmol), cyanamide (0.13 g, 3.1 mmol) in ethyl acetate (5 ml) was added 1 drops of concentrated HCl with magnetic stirring. The reaction mixture was then refluxed for 5 hr. After cooling to room temperature, the solvent was evaporated in vacuo and extracted with ethyl acetate and NaHCO₃ solution. The organic layer was then dried over Na₂SO₄, filtered and concentrated to provide compound 7f (0.02 g, 13%) as pale orange powders.

Compound 7f: Mwt: 345.15; R_{f}: 0.50 (ethyl acetate); mp: 238-240° C.; UV (MeOH): λₘₐₓ nm (log ε)=217 (1.14). HRMS (EI) m/z (%): calcd.: 344.0120 (M⁺). found: 283.9799 (M-60), 240.0295 (100). ¹H-NMR (CD₃OD, 300 MHz) δ (ppm): 2.57 (3H, s, CH₃), 4.00 (3H, s, OCH₃), 7.96 (1H, s, Ar H-6), ¹³C-NMR (DMSO-d₆, 75 MHz) δ (ppm): 14.49, 52.68, 104.56, 107.70, 114.75, 132.17, 141.00, 151.65, 152.70, 155.60, 170.38.

### Example 17

### Synthesis of 4-(acetamido)-5-amino-3-bromo-2-hydroxybenzoic acid (compound 8)

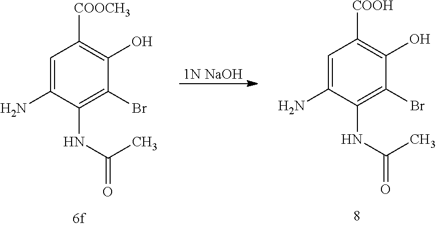

To a mixture of compound 6f (0.3 g, 1 mmol) in 1N NaOH (10 ml) was stirred with magnetic stiffing overnight. After acidified with 1N HCl to pH˜6, the solvent was evaporated in vacuo. The resulting residue was dissolved in MeOH (20 ml) and filtrated. The collected filtrate was concentrated and collected the precipitates to provide compound 8 as beige powders (260 mg, 90% yield).

Compound 8: Mwt: 289.09; R_{f}: 0.36 (ethyl acetate/methanol=2:1); mp: 210-211° C. HRMS (EI) m/z (%): calcd.: 289.0871 (M⁺). found: 271.0819 (M-18), 185.0795 (100). ¹H-NMR (DMSO-d₆, 300 MHz) δ (ppm): 2.19 (3H, s, CH₃), 7.74 (1H, s, Ar H-6), 10.34 (1H, s, ArNH).

### Example 18

### Anti-Influenza Assay

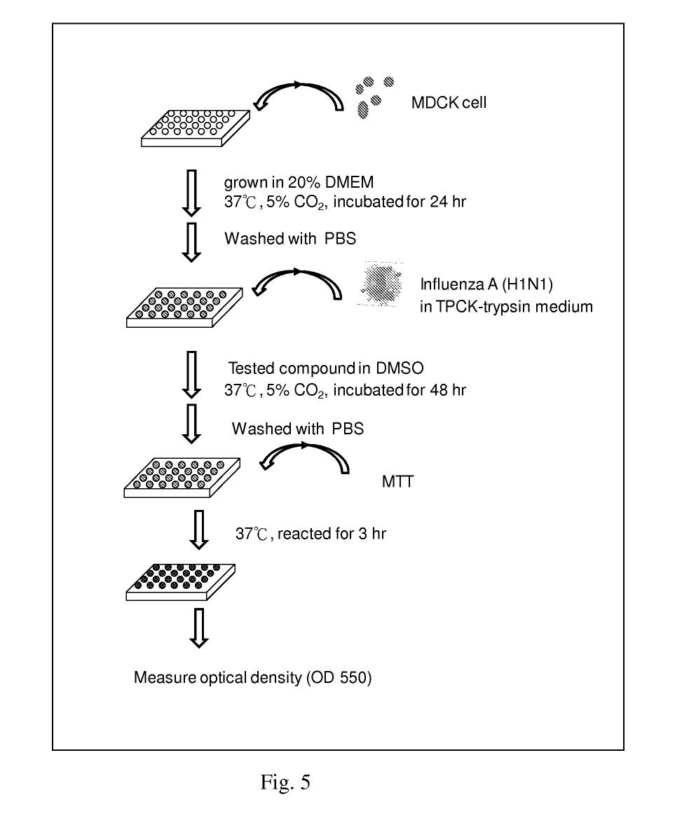

This experiment was performed by the division of clinical pathology tri-service general hospital, and the procedure was described as follows (as shown in FIG. 5).

### 1. Cell Culture

The cell line used in this experiment was MDCK cells (Madin-Darby canine kidney). MDCK cells were incubated with DMEM (Dulbecco's modified Eagle's medium) containing 10% FBS (Fetal bovine serum) in a 96-well microplate for 24 hr at 37° C., 5% CO₂.

### 2. Adding Drugs and Viruses

While cells grown to 70-80% confluence, the cells in each well were washed with PBS (phosphate buffered saline) and divided into 3 groups: (i) blank group: MDCK cells were cultured in TPCK-trypsin medium without drugs and viruses; (ii) D group: MDCK cells were cultured in TPCK-trypsin medium added with 100 μL drug; (iii) DV group: MDCK cells were cultured in TPCK-trypsin medium added with 50 μL virus-containing supernatant and 100 μL drug. The groups were incubated in an incubator for 48 h at 37° C., 5% CO₂.

MDCK cells were infected with H₁N₁ virus at 0.01 MOI (multiplicity of infection, MOI=virus particles/number of infected cells) in TPCK-trypsin medium. Tested compounds were dissolved in DMSO (dimethyl sulfoxide), and then were series diluted to 100, 50, 25, 12.5, 6.25, 3.12, 1.56, 0.78 and 0.39 μg/ml, respectively.

### 3. Analysis of Cell Viability and Inhibition of Viral Activity

MTT agent was added and the cells were incubated in dark for 5 h. After incubation, supernatant was removed and DMSO was added to dissolve formazan products, and the plate was incubated in an incubator for 10-15 min. Each well was measured by an ELISA reader at O.D. 550 nm.

By use of cell viability obtained by MTT assay, MIC (Minimum inhibitory concentration and CC₅₀ (The 50% cell cytotoxic concentration) were also calculated.

MTT (Methylthiazoleltetrazolium bromide), a yellow water soluble organic dye, could react with succinate dehydrogenase of mitochondria in living cells to convert tetrazolium to formazan (purple crystal product). Formazan products were in direct ratio to living cells, which could be used to estimate the cell viability. Cell viability formulation was shown as follow:

Cell viability (%)=(OD₅₅₀ of drug-treated well−OD₅₅₀ of blank group)/(OD₅₅₀ of negative control−OD₅₅₀ of blank group)×100%

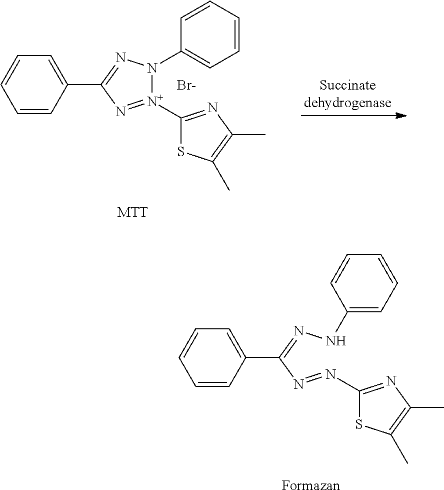

### RESULTS

Totally 11 compounds of the present invention were obtained, and compounds 6a, 6b, 6c, 6d, 6e, 6f, 7a, 7b, 7e, 7f and 8 were screened for their pharmaceutical activity. The compounds were dissolved in DMSO and diluted to 100, 50, 25, 12.5, 6.25, 3.12, 1.56, 0.78 and 0.39 μg/ml for pharmaceutical activity screening. The experiment was done in triplicate, and the initial results were shown as table 5:

As Table 5 shown, all compounds (<100 μg/ml) were non-toxic to MDCK cells. Compounds 6a, 6b, 6c, 6d, 6e, 6f, 7a, 7b and 8 had better anti-H1N1 activity. In particular, MIC of compound 6a was 50 μg/mL, and its SI (selective index=CC₅₀/MIC) was higher than 2; MIC of compound 6b was 1.56 μg/mL, and its SI was higher than 64; MIC of compound 6c was 25 μg/mL, and its SI was higher than 4; compound 6d has activities as same as compound 6b; the most potent of them is compound 6f that has MIC 0.78 μg/mL and SI 128; MIC of compound 7a was 25 μg/mL, and its SI was higher than 4; MIC of compound 7b was 25 μg/mL, and its SI was higher than 4; MIC of compound 8 was 1.56 μg/mL and its SI was higher than 64. However, compounds 7e and 7f were shown no inhibiting activity.

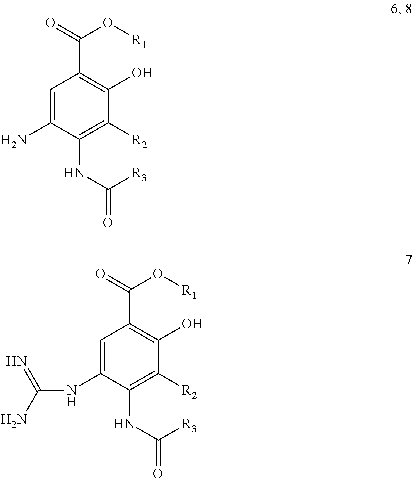

TABLE 5
Anti-influenza virus (H1N1) activities of 6 and 7 in MDCK cell

| | | | | CC₅₀ | MIC | |
|---|---|---|---|---|---|---|
| compound | R₁ | R₂ | R₃ | (μg/ml) | (μg/ml) | SI |
| 6a | H | H | CH₃ | >100 | 50 | >2 |
| 6b | CH₃ | H | CH₃ | >100 | 1.56 | >64 |
| 6c | H | H | C₃H₇ | >100 | 25 | >4 |
| 6e | C₂H₅ | H | C₃H₇ | >100 | 1.56 | >64 |
| 6f | CH₃ | Br | CH₃ | >100 | 0.78 | >128 |
| 7a | H | H | CH₃ | >100 | 25 | >4 |
| 7b | C₂H₅ | H | C₃H₇ | >100 | 25 | >4 |
| 7e | C₂H₅ | H | C₃H₇ | >100 | >100 | ND |
| 7f | CH₃ | Br | CH₃ | >100 | >100 | ND |
| 8 | H | Br | CH₃ | >100 | 1.56 | |
| PAS | | | | >100 | >100 | ND |
| Ribavirin | | | | >100 | 1.56 | >64 |

EC₅₀: the 50% effective concentration,
CC₅₀: the 50% cell cytotoxic concentration,
MIC: minimum inhibitory concentration (cell survival rate >75%),
ND: not determined.
PAS: p-aminosalicylic acid.

According to table 5, the compounds of the present invention (compounds 6a, 6b, 6c, 6d, 6e, 6f, 7a, 7b and 8) could effectively inhibit influenza viral activity. Particularly, compounds 6b, 6d, 6e, 6f and 8 had preferred anti-influenza activity, and these compounds were suitable to be added to a pharmaceutical composition. In addition to the foregoing compounds, the pharmaceutical composition could comprise a pharmaceutical acceptable carrier, such as, but not limited to, an excipient (e.g. water), a filler (e.g. sucrose or starch), an adhesive (e.g. cellulose derivatives), a diluent agent, a disintegration agent, a delivery agent, or a sweetening-agent. The pharmaceutical composition could be produced by traditional manufactures, and its dosage form could be made by mixing an effective amount of the compounds with at least one carrier to generate the required dosage form, which comprised, but not limited to, tablet, pellet, powder, capsule and other liquid dosage forms.

As the results exhibited in anti-influenza assay, 4,5-diamino-3-halo-2-hydroxybenzoic acid derivatives provided here were non-toxic to MDCK cells, particularly compounds 6a, 6b, 6c, 6e, 6f, 7a, 7b and 8 had better anti-H1N1 activity. In the future, these compounds can focus on viral neuraminidases as targets to develop effective anti-influenza drugs.

What is claimed is:

1. A 4,5-diamino-3-halo-2-hydroxybenzoic acid derivative, as presented by formula (I): 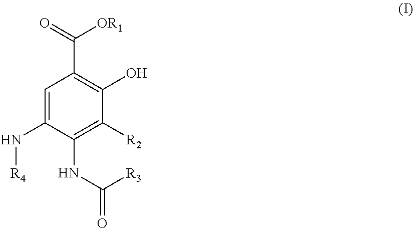
wherein R₁ group is H, CH₃, or C₂H₅;
R₂ group is H, or Br;
R₃ group is CH₃, or C₃H₇; and
R₄ group is H, or C(═NH)—NH₂.

2. The 4,5-diamino-3-halo-2-hydroxybenzoic acid derivative as claimed in claim 1, which is selected from the group consisting of 4-(acetamido)-5-amino-2-hydroxybenzoic acid (compound 6a), methyl 4-(acetamido)-5-amino-2-hydroxybenzoate (compound 6b), 5-amino-4-(butyramido)-2-hydroxybenzoic acid (compound 6c), methyl 5-amino-4-(butyramido)-2-hydroxybenzoate (compound 6d), ethyl 5-amino-4-(butyramido)-2-hydroxybenzoate (compound 6e), methyl 4-(acetamido)-5-amino-3-bromo-2-hydroxybenzoate (compound 6f). 4-(acetamido)-5-guanidino-2-hydroxybenzoic acid (compound 7a), methyl 4-(acetamido)-5-guanidino-2-hydroxybenzoate (compound 7b), ethyl 4-(butyramido)-5-guanidino-2-hydroxybenzoate (compound 7e), methyl 4-(acetamido)-3-bromo-5-guanidino-2-hydroxybenzoate (compound 7f) and 4-(acetamido)-5-amino-3-bromo-2-hydroxybenzoic acid (compound 8).

3. The 4,5-diamino-3-halo-2-hydroxybenzoic acid derivative as claimed in claim 1, which is selected from the group consisting of 4-(acetamido)-5-amino-2-hydroxybenzoate (compound 6b), methyl 5-amino-4-(butyramido)-2-hydroxybenzoate (compound 6d), ethyl 5-amino-4-(butyramido)-2-hydroxybenzoate (compound 6e), methyl 4-(acetamido)-5-amino-3-bromo-2-hydroxybenzoate (compound 6f) and 4-(acetamido)-5-amino-3-bromo-2-hydroxybenzoic acid (compound 8).

4. An anti-influenza pharmaceutical composition, comprising the 4,5-diamino-3-halo-2-hydroxybenzoic acid derivative as claimed in claim 1 or the pharmaceutically acceptable salts thereof.

5. A method of treating influenza caused by a virus, comprising administrating to a subject suffering from influenza with an effective amount of a 4,5-diamino-3-halo-2-hydroxybenzoic acid derivative as claimed in claim 1.

6. A method of treating influenza caused by a virus, comprising administrating to a subject suffering from influenza with an effective amount of a 4,5-diamino-3-halo-2-hydroxybenzoic acid derivative as claimed in claim 2.

7. A method of treating influenza caused by a virus, comprising administrating to a subject suffering from influenza with an effective amount of a 4,5-diamino-3-halo-2-hydroxybenzoic acid derivative as claimed in claim 3.

8. The method as claimed in claim 5, wherein the 4,5-diamino-3-halo-2-hydroxybenzoic acid derivative is adapted to inhibit influenza neuraminidase of the subject.

9. The method as claimed in claim 6, wherein the 4,5-diamino-3-halo-2-hydroxybenzoic acid derivative is adapted to inhibit influenza neuraminidase of the subject.

10. The method as claimed in claim 7, wherein the 4,5-diamino-3-halo-2-hydroxybenzoic acid derivative is adapted to inhibit influenza neuraminidase of the subject.

11. The method as claimed in claim 5, wherein the influenza is type A influenza or type B influenza.

12. The method as claimed in claim 6, wherein the influenza is type A influenza or type B influenza.

13. The method as claimed in claim 7, wherein the influenza is type A influenza or type B influenza.

14. The method as claimed in claim 5, wherein the virus is influenza virus type A subtype H1N1.

15. The method as claimed in claim 6, wherein the virus is influenza virus type A subtype H1N1.

16. The method as claimed in claim 7, wherein the virus is influenza virus type A subtype H1N1.

17. A method of manufacturing the 4,5-diamino-3-halo-2-hydroxybenzoic acid derivatives as claimed in claim 1, comprising the steps of: providing p-aminosalicylic acid (PAS) as a initial reagent, wherein the carboxyl group on C-1 site is kept or converted to an ester group; the hydrogen atom on C-3 site is kept or converted to a bromo group, the amino group on C-4 site is converted to a amido group, the hydrogen atom on C-5 site is converted to amino or guanidino group; 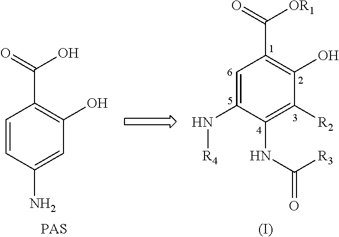
wherein R₁ group is H, CH₃, or C₂H₅;
R₂ group is H, or Br;
R₃ group is CH₃, or C₃H₇; and
R₄ group is H, or C(═NH)—NH₂.

18. A method of manufacturing the 4,5-diamino-3-halo-2-hydroxybenzoic acid derivatives as claimed in claim 2, comprising the steps of: providing p-aminosalicylic acid (PAS) as a initial reagent, wherein the carboxyl group on C-1 site is kept or converted to an ester group; the hydrogen atom on C-3 site is kept or converted to a bromo group, the amino group on C-4 site is converted to a amido group, the hydrogen atom on C-5 site is converted to amino or guanidino group; 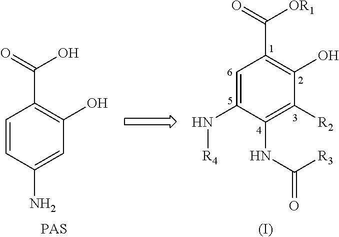
wherein R₁ group is H, CH₃, or C₂H₅;
R₂ group is H, or Br;
R₃ group is CH₃, or C₃H₇; and
R₄ group is H, or C(═NH)—NH₂.

19. A method of manufacturing the 4,5-diamino-3-halo-2-hydroxybenzoic acid derivatives as claimed in claim 3, comprising the steps of: providing p-aminosalicylic acid (PAS) as a initial reagent, wherein the carboxyl group on C-1 site is kept or converted to an ester group; the hydrogen atom on C-3 site is kept or converted to a bromo group, the amino group on C-4 site is converted to a amido group, the hydrogen atom on C-5 site is converted to amino or guanidino group; 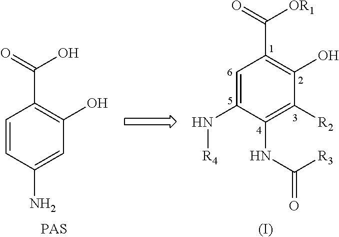
wherein R₁ group is H, CH₃, or C₂H₅;
R₂ group is H, or Br;
R₃ group is CH₃, or C₃H₇; and
R₄ group is H, or C(═NH)—NH₂.

20. The method as claimed in claim 17, wherein the 4, 5-diamino-3-halo-2-hydroxybenzoic acid derivative is methyl 4-(acetamido)-5-amino-3-bromo-2-hydroxybenzoate (compound 6f).

21. The method as claimed in claim 18, wherein the 4, 5-diamino-3-halo-2-hydroxybenzoic acid derivative is methyl 4-(acetamido)-5-amino-3-bromo-2-hydroxybenzoate (compound 6f).

22. The method as claimed in claim 19, wherein the 4, 5-diamino-3-halo-2-hydroxybenzoic acid derivative is methyl 4-(acetamido)-5-amino-3-bromo-2-hydroxybenzoate (compound 6f).

23. The method as claimed in claim 20, further comprising a step of adding the methyl 4-(acetamido)-5-amino-3-bromo-2-hydroxybenzoate (compound 6f) to NaOH solution to form 4-(acetamido)-5-amino-3-bromo-2-hydroxybenzoic acid (compound 8).

24. The method as claimed in claim 21, further comprising a step of adding the methyl 4-(acetamido)-5-amino-3-bromo-2-hydroxybenzoate (compound 6f) to NaOH solution to form 4-(acetamido)-5-amino-3-bromo-2-hydroxybenzoic acid (compound 8).

25. The method as claimed in claim 22, further comprising a step of adding the methyl 4-(acetamido)-5-amino-3-bromo-2-hydroxybenzoate (compound 6f) to NaOH solution to form 4-(acetamido)-5-amino-3-bromo-2-hydroxybenzoic acid (compound 8).
